(19) United States
(12) Patent Application Publication
(10) Pub. No.: US 2026/0364093 A1
(43) Pub. Date: Nov. 19, 2026

(54) Aza-Platinum-Dipyrromethene Anti-Cancer Agents

(71) Applicant: MCPHS University, Boston, MA (US)

(72) Inventors: Ronny PRIEFER, Hopkinton, MA (US); Michelle Young, Belmont, MA (US); Andi Zeng, Quincy, MA (US)

(21) Appl. No.: 19/667,236

(22) Filed: May 4, 2026

Related U.S. Application Data
(60) Provisional application No. 63/799,318, filed on May 2, 2025.

(51) Int. Cl. A61K 31/555; A61P 35/00; C07F 15/00; C07K 16/22; C07K 16/28; C07K 16/32

(52) U.S. Cl. CPC A61K 31/555; A61P 35/00; C07F 15/0086; C07K 16/22; C07K 16/2863; C07K 16/2887; C07K 16/32

(57) ABSTRACT

Aza-platinum-dipyrromethenes (aza-PLADIPYs) are a novel class of platinum-containing anticancer agents. Aza-PLADIPY compounds produce DNA crosslinking-based chemotoxicity. The compounds showed potent cytotoxicity on ovarian cancer cell lines and other cancer cell lines. Cytotoxicity surpassed that of cisplatin with virtually no nephrotoxicity. Thus, the aza-PLADIPY compounds offer significant improvement over the dose-limiting nephrotoxic side effects of platinum containing anticancer agents.

### CROSS REFERENCE TO RELATED APPLICATIONS

This application claims the priority of U.S. Provisional Application No. 63/799,318 filed 2 May 2025 and entitled “Aza-Platinum-Dipyrromethene Anti-Cancer Agents”, the whole of which is hereby incorporated by reference.

### BACKGROUND

The targets of anticancer agents include alkylating agents, mitotic inhibitors, and kinase antagonists.¹ One approach to chemotherapeutic cancer treatment is the use of cross-linking agents that target the DNA of rapidly dividing cells.¹ This class of anticancer agents is subdivided into organic agents (e.g. nitrogen mustard, Mitomycin C) and inorganic agents (e.g. cisplatin). Regardless of the subdivision, these agents target the N7 of guanine and will crosslink DNA, either inter- or intra-strand, and will also form crosslinks in proteins. Crosslinking of DNA prevents the reading of the DNA during both replication and transcription.²

Another approach for targeting cancer is to utilize photodynamic therapy (PDT). With PDT, a photosensitizer (PS) is irradiated with light at a specific wavelength, causing it to undergo an S₀ to S₁ excitation, followed by intersystem crossing to its T₁ state, which subsequently donates its energy to O₂ to produce a plethora of reactive oxygen species (ROS). This can trigger necrotic, apoptotic, or other forms of cell death.³ Examples of PDT agents used in cancer treatment include: PHOTOFRIN (used in esophageal and non-small cell lung cancer)⁴, aminolevulinic acid (used in actinic keratosis)⁴, temoporfin (used to treat head and neck cancer).⁵ PHOTOFRIN was the first PS approved by the FDA for clinical use.⁶ It is a porphyrin-containing PS and shows a strong absorption band at ~400 nm, and a weaker band between 500 to 650 nm.⁷ Some recent PSs that have been reported are the boron possessing aza-boron-dipyrromethenes (aza-BODIPYs). The PHOTOFRIN-like attachment to the boron core allows for a shifting of absorbance so that deeper tumor targeting is possible.⁸

Although these approaches are effective and light activation confers spatial selectivity with minimal off-target effects, PS have their own set of limitations, such as low bioavailability, inability of light to penetrate to the tumor site, as well as being ineffective in the hypoxic conditions of some tumor cells.³ Conversely, the DNA crosslinking agent, cisplatin, is known to cause severe, dose-limiting nephrotoxicity, in addition to gastrointestinal damage and neurotoxicity.⁹ There is a need for anticancer agents with high but selective cytotoxicity for cancer cells, and low toxicity to non-cancer cells.

### SUMMARY

The present technology provides a novel class of platinum-containing anticancer agents, specifically aza-platinum-dipyrromethenes (aza-PLADIPYs). The compounds produce DNA crosslinking-based chemotoxicity. The aza-PLADIPYs were found to possess a distorted square planar orientation with an unexpected Pt—C bond being generated. The activity of these agents was compared to PHOTOFRIN (a photodynamic therapy (PDT) agent) and cisplatin (a DNA crosslinking agent). Although the aza-PLADIPY compounds did not possess any PDT activity, they showed potent cytotoxicity on OVCAR 5 cells. Compound 4 (FIG. 1, R=OCH₃) displayed the highest cytotoxicity (about 78% cell death at 250 μM), surpassing that of cisplatin (about 54% cell death at 250 μM). Impressively, the aza-PLADIPYs are expected to show virtually no nephrotoxicity at concentrations up to 250 μM, based on testing performed on HK-2 kidney proximal tubule cells. Thus, the aza-PLADIPY compounds offer significant improvement over the dose-limiting nephrotoxic side effects of platinum containing anticancer agents.

The present technology can also be summarized with the following listing of features.
1. A compound of Formula I:

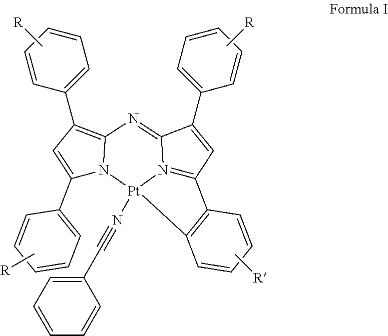

wherein R and R′ are independently selected from one or more substituents on a designated ring structure, the substituents selected from the group consisting of H, C1-C12 linear or branched alkyl, C1-C12 alkylamino, amino, hydroxy, C1-C12 hydroxyalkyl, C1-C12 thioalkyl, C1-C12 cycloalkyl, alternatively C3-C12 cycloalkyl, aryl, C1-C12 alkoxy, C1-C12 amide, halo, C1-C12 haloalkyl, CF₃, NO₂, cyano, C1-C6 carboxy, and C1-C12 sulfonate, wherein R and R′ are ortho, meta, or para.
2. The compound of feature 1, wherein R is methyl, methoxy, or chloro.
3. The compound of feature 1, wherein R and/or R′ is methoxy.
4. The compound of feature 1, wherein the compound is selected from the following compounds:

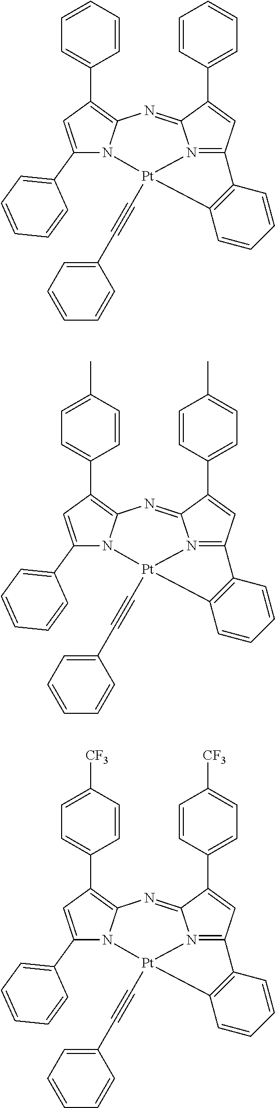

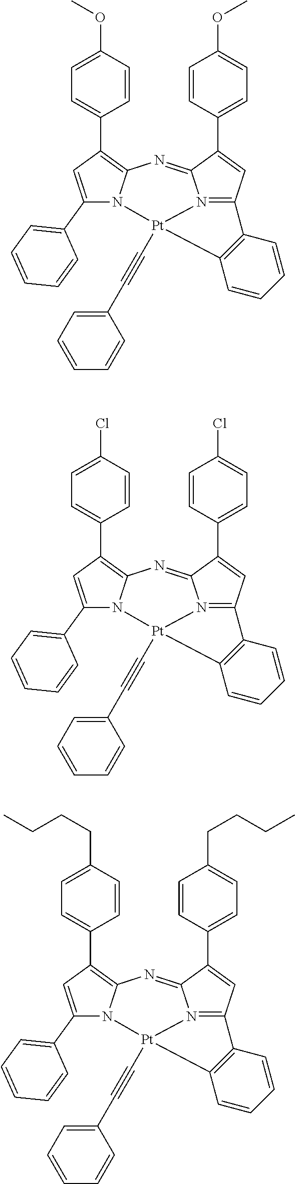

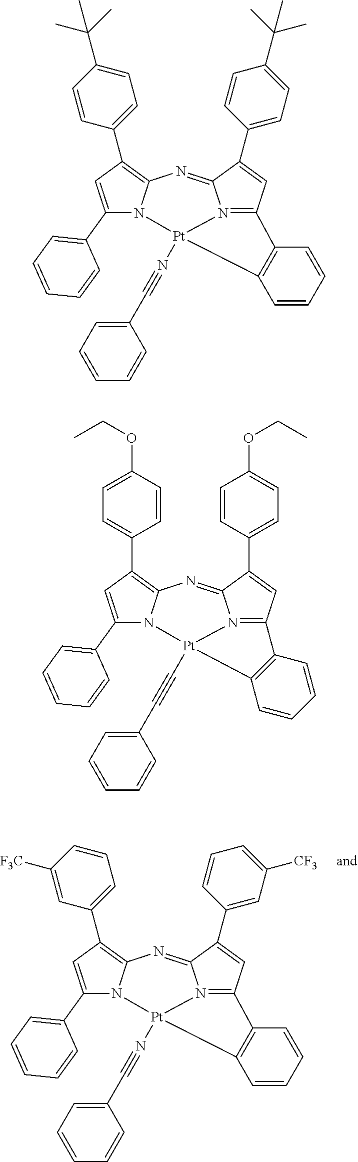

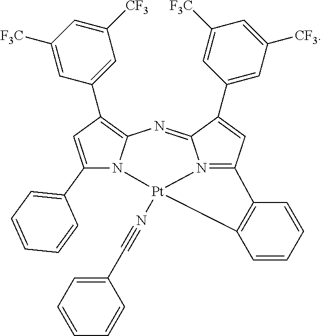

5. The compound of any of the preceding features, wherein the compound is an anticancer agent.
6. The compound of any of the preceding features, wherein the compound has a higher cytotoxicity toward cancer cells than cisplatin.
7. The compound of any of the preceding features, wherein the compound kills at least 70% of cancer cells at a concentration of 250 M.
8. The compound of any of the preceding features, wherein the compound lacks nephrotoxicity at a concentration that produces at least 50% cell death of cancer cells.
9. A pharmaceutical composition comprising the compound of any of the preceding features and at least one excipient.
10. The pharmaceutical composition of feature 9, further comprising one or more additional antitumor agents.
11. The pharmaceutical composition of feature 10, wherein the one or more additional antitumor agents are selected from small molecule antitumor agents, aptamers and antibodies.
12. The pharmaceutical composition of feature 11, wherein the one or more additional antitumor agents are small molecules selected from the group consisting of alkylating agents, antimetabolites, antibiotics, taxanes, Vinca alkaloids, kinase inhibitors, and immune checkpoint inhibitors.
13. The pharmaceutical composition of feature 11, wherein the one or more additional antitumor agents are antibodies selected from the group consisting of rituximab, trastuzumab, bevacizumab, cetuximab.
14. The pharmaceutical composition of feature 11, wherein the one or more additional antitumor agents are antibodies selected from the group consisting of monoclonal antibodies, bispecific antibodies, and recombinant single chain antibodies.
15. A method of treating cancer in a mammalian subject, the method comprising administering to a mammalian subject in need thereof the compound of any of features 1-8 or the pharmaceutical composition of any of features 9-14, whereby cancer cells in the subject are killed.
16 The method of feature 15, wherein said administering is not nephrotoxic.
17 The method of feature 15 or 16, wherein the cancer is selected from the group consisting of testicular cancer, ovarian cancer, bladder cancer, lung cancer, head and neck cancer, endometrial cancer, gastric cancer, esophageal cancer, breast cancer, cervical cancer, prostate cancer, neuroblastoma, multiple myeloma, melanoma, mesothelioma, leukemia, non-Hodgkin's lymphoma, and sarcomas.
18. The method of any of features 15-17, wherein said compound or pharmaceutical composition is administered by intravenous administration.
19. The method of any of features 15-18, further comprising administering radiation treatment to the mammalian subject.
20. A pharmaceutical kit or pack comprising the compound of any of features 1-8 or the pharmaceutical composition of any of features 9-14 and another pharmaceutically active agent.

### BRIEF DESCRIPTION OF THE DRAWINGS

FIG. 1 shows a reaction scheme for the synthesis of aza-PLADIPY Compounds 1-5.

FIG. 2 shows the structure of Compound 4 as determined by X-ray diffraction.

FIG. 3 shows the viability of OVCAR 5 cells in the presence of 250 μM of the indicated control compounds and aza-PLADIPY Compounds 1-5, performed in the dark or under white light as indicated. Results are normalized to vehicle control.

FIGS. 4A and 4B show the viability of OVCAR5 cells in the presence of 100 (FIG. 4A) or 250 μM (FIG. 4B) of the indicated control compounds and aza-PLADIPY Compounds 1-5, performed in the dark or under white light as indicated. Results are normalized to vehicle control.

FIG. 5 shows a reaction scheme for the synthesis of aza-PLADIPY Compounds 6-10.

FIG. 6 shows the viability of HK-2 kidney cells in the presence of the indicated concentrations of Compounds 1, 2, and 4 compared to cisplatin.

FIG. 7 illustrates the structures of Compounds 6-10.

FIG. 8 presents a summary of OVCAR 5 cell viability data for Compounds 6-10 compared to cisplatin.

FIGS. 9A-9C show dose-response curves for OVCAR 5 cell viability for Compounds 6 and 7 compared to cisplatin.

### DETAILED DESCRIPTION

The present technology provides a class of antitumor compounds which are platinum derivatives of aza-BODIPY analogs (BODIPY refers to boron dipyrromethene). Specifically, the compounds are based on aza-platinum-dipyrromethene, and are herein referred to as aza-PLADIPYs. Regarding the stereochemistry of the aza-BODIPYs, trans-platin is relatively inactive compared to its cis counterpart.¹⁰ The planar nature of DIPY, coupled with its high degree of conjunction, allows for intercalation into the DNA strand. The aza-PLADIPYSs thus provide effective and versatile chemotherapeutic agents.

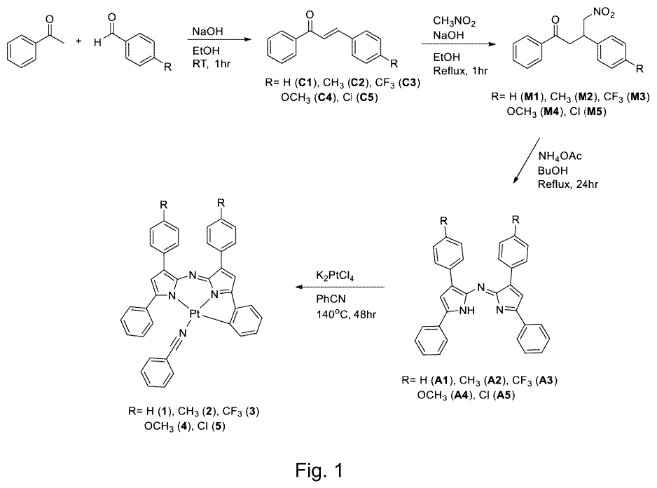

The synthesis of azadipyrromethene derivatives was carried out following the methodology described by Priefer⁸ (FIG. 1). This involved the initial chalcone synthesis of a benzaldehyde and an acetophenone to generate C1-5, followed by the synthesis of Micheal's adduct (M1-5). Subsequent treatment with ammonium acetate in butanol with refluxing for 24 hours, afforded the azadipyrromethene ligands (A1-5). Several methods of coordinating the platinum into the core were evaluated using general procedure for coordinating nitrogen to platinum.^{11,12} These procedures were derived from the synthesis of cis-platin and required both a platinum salt and a ligand that is water soluble. Since the azadipyrromethene ligands are water insoluble, the procedure was modified and performed in water and a water miscible co-solvent, such as THF, DMSO, EtOH, or ACN. However, the ligand was only sparingly soluble in these solvent systems. Heating to reflux did not appear to improve solubility to any appreciable amount and monitoring with TLC showed no formation of any new material. Use of aqueous hydroxides (i.e. NaOH, KOH) or amine bases (i.e. Et₃N, DIPEA) did not result in product.

A different approach was to use a platinum reagent that is soluble in organic solvents. K₂PtCl₄ can be converted into cis-dichlorobis(dimethyl sulfoxide) platinum (II), 13 which is soluble in most organic solvents, including CH₂Cl₂, the solvent commonly used to synthesize aza-BODIPY.⁸ However, no reaction occurred between the azadipyrromethene and the dichlorobis(dimethylsulfoxide) platinum (II), whether at room temperature or under reflux conditions.

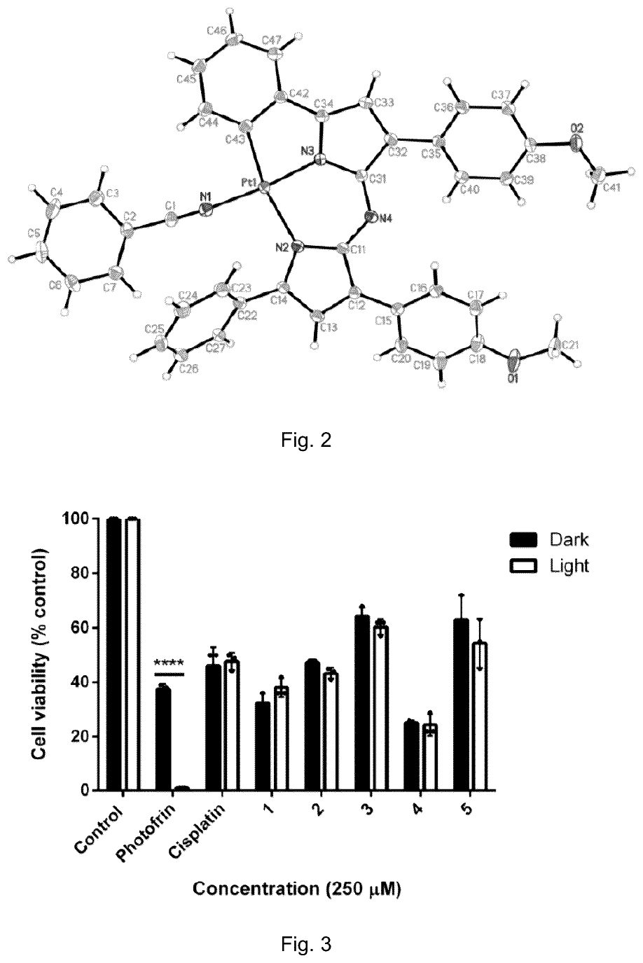

The successful procedure involved the heating of the azadipyrromethene ligand with K₂PtCl₄ in benzonitrile at 140 °C for two days. Silica gel column chromatography was used for purification of the crude and 1H-NMR of the product revealed an unexpected 1,2-disubstituted benzene ring pattern. The formation of this new bond seems to be unprecedented and NMR alone could not determine the coordination structure of this molecule. Ultimately, X-ray crystallography (see FIG. 2) confirmed it to be a cyclometallated platinum azadipyrromethene (vide infra).

Using the same procedure, a total of five novel aza-PLADIPYs (1-5) were synthesized, with yields ranging from 20-40%. Extending the reflux time consumed more starting material, but also led to the formation of numerous unisolated by-products. Two days of heating was deemed the optimal reaction time, based on monitoring with TLC and NMR.

The Pt^{II} complex displays a distorted square planar coordination (FIG. 1). For the adjacent atoms surrounding the Pt core, the average bond angles across the five aza-PLADIPYs were: 87° [N(3)-Pt(1)-N(2)], 100° [N(2)-Pt(1)-N(1)], 80° [N(1)-Pt(1)-C(43)], and 81° [C(43)-Pt(1)-N(3)]. For the trans-positioned atoms across the Pt core, the average bond angles were: 172° [N(3)-Pt(1)-N(1)] and 167° [N(2)-Pt(1)-C(43)]. Additionally, the average bond length for the Pt(1)-N(3) bond was 1.95 Å, Pt(1)-N(2): 2.12 Å, Pt(1)-N(1): 1.97 Å, and Pt(1)-C(42): 2.21 Å. The latter Pt—C bond length falls within the reported literature range of 2.10-2.33 Å.¹⁷

The effectiveness of the novel aza-PLADIPYs as anticancer agents was tested. Platinum-containing compounds are often the first line treatment for high-grade serous ovarian cancer.¹⁸ However, chemoresistance is a major challenge in this therapeutic approach.¹⁸ To address this issue, a cell line was chosen that demonstrates sensitivity to platinum-containing chemotherapeutic agents while also exhibiting some level of resistance to cisplatin. This would help reveal whether the synthesised aza-PLADIPYs show efficacy in a cell line with partial cisplatin resistance.¹⁹ As a result the OVCAR5 cell line was selected to test our aza-PLADIPYs.¹⁹ Since it was hypothesised that the synthesised compounds could possess a dual mechanisms of action (i.e. DNA crosslinking and phototoxicity) two positive controls were used to assess each effect. Cisplatin, the platinum containing DNA crosslinking agent, was used as a positive control to assess DNA crosslinking; whereas, Photofrin® was used to assess phototoxicity. The synthesized compounds, as well as the positive controls, were tested at two different concentrations, namely 100 and 250 μM. The cells were incubated with either the positive controls or the synthesized compounds for 1 hour, after which a light dose (either white light, 635 nm, or 690 nm) was administered to half of the cells to assess the potential to produce phototoxicity. Conversely, the other half was left in darkness to assess chemotoxicity. Following treatment and light dosing, cells were incubated for 24 hours, followed by determining of cell viability (Table 1).

TABLE 1
Percentage of cell death caused by Photofrin ®, cisplatin and Compounds
1-5 across three different light sources and in dark conditions (n = 3).

| | | | | | | | | Average |
|---|---|---|---|---|---|---|---|---|
| | | % Cell death in respective light source | | | | | | % Cell |
| | Concentration | White Light | | 635 nm | | 690 nm | | death |
| Compounds | in μM | LIGHT | DARK | LIGHT | DARK | LIGHT | DARK | (DARK) |
| Photofrin ® | 100 | 98 | 39 | 76 | 62 | 23 | 36 | 45 |
| | 250 | 99 | 56 | 83 | 74 | 42 | 47 | 59 |
| Cisplatin | 100 | 48 | 44 | 45 | 45 | 40 | 54 | 47 |
| | 250 | 53 | 55 | 54 | 55 | 51 | 54 | 54 |
| 1 | 100 | 37 | 21 | 22 | 35 | 20 | 31 | 29 |
| | 250 | 62 | 69 | 69 | 62 | 65 | 67 | 65 |
| 2 | 100 | 31 | 16 | 21 | 27 | 16 | 23 | 22 |
| | 250 | 58 | 53 | 51 | 56 | 50 | 54 | 54 |
| 3 | 100 | 7 | 12 | 14 | 9 | 1 | 10 | 10 |
| | 250 | 40 | 36 | 37 | 40 | 29 | 39 | 38 |
| 4 | 100 | 46 | 51 | 50 | 45 | 48 | 51 | 49 |
| | 250 | 77 | 76 | 77 | 78 | 81 | 82 | 78 |
| 5 | 100 | 14 | 14 | 12 | 15 | 13 | 12 | 13 |
| | 250 | 46 | 37 | 40 | 47 | 40 | 50 | 45 |

At a dose of 100 μM of PHOTOFRIN, 98% cell death in white light and an average of 45% in the dark was observed. At 250 μM, 99% cell death in white light and an average of 59% in dark conditions was seen (FIG. 3). Similar to cisplatin, the aza-PLADIPYs produced no significant differences in cell viability between light and dark conditions. This indicated that the novel aza-PLADIPYs did not act as PSs at either concentration tested or light source employed, and thus would not have the potential for use in PDT.

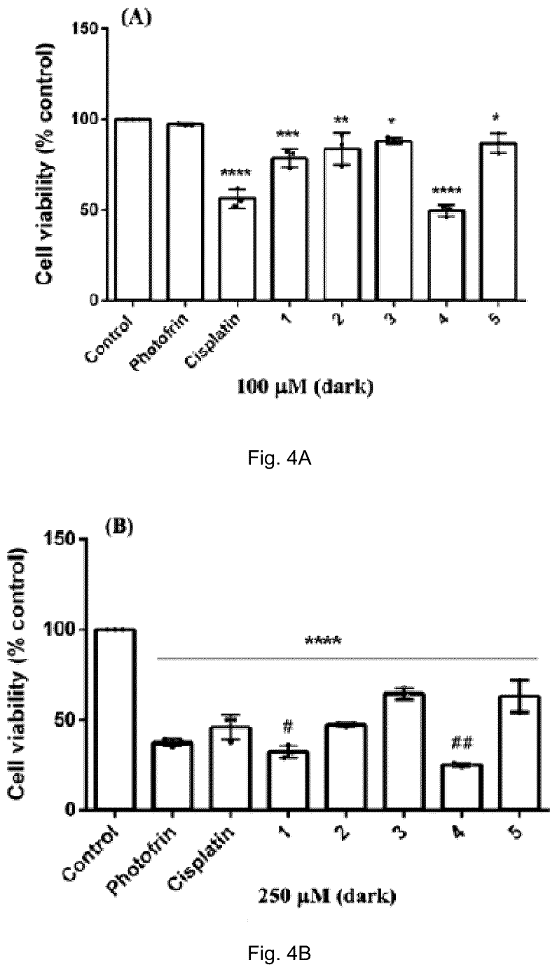

The aza-PLADIPYs were compared to the DNA crosslinker, cisplatin. At 100 μM, cisplatin produced ~47% cell death (FIG. 4A). At the same concentration Compound 1 (R=H, ~29% cell death), 2 (R=CH₃, ~22% cell death), 3 (R=CF₃, ~10% cell death) and 5 (R=Cl, ~13% cell death) displayed lower cytotoxicity than cisplatin. Compound 4 (R=OCH₃, ~49% cell death) had comparable efficacy to that of cisplatin. At 250 μM, cisplatin displayed ~54% cell death (FIG. 4B). At this same concentration, Compounds 3 (R=CF₃, ~38% cell death) and 5 (R=Cl, ~45% cell death) had slightly lower cell death compared to cisplatin. Compound 1 (R=H, ~65% cell death) and 2 (R=CH₃, ~54% cell death) showed statistically comparable cytotoxicity to cisplatin. Most notably, Compound 4 (R=OCH₃, ~78% cell death) displayed statistically superior cytotoxicity compared to cisplatin.

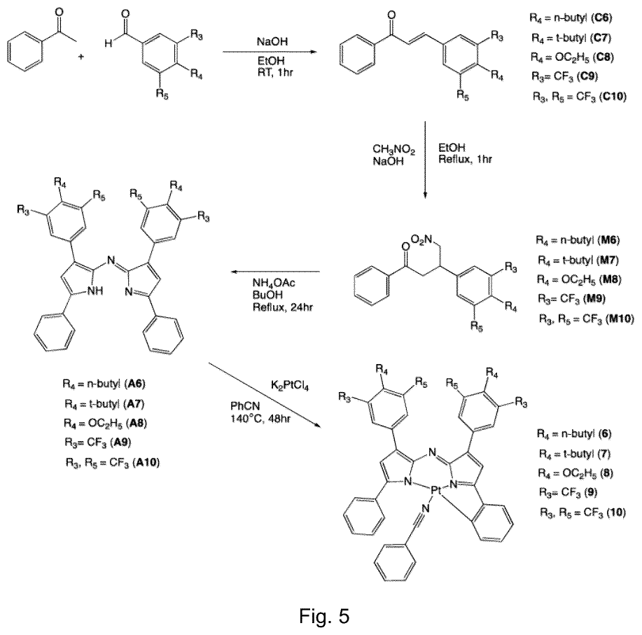

As discussed previously, cisplatin has a notable side effect of nephrotoxicity, which produces acute kidney injury in 20-30% of patients.²⁰ This damage is a result of accumulation of cisplatin in the kidney's proximal tubules, resulting in oxidative stress and inflammation, ultimately leading to cell death.²¹ As a result, the compounds that displayed comparable or superior cytotoxicity than cisplatin (i.e. Compounds 1, 2, and 4) were tested on HK-2 kidney proximal tubule cells. Cisplatin and the synthesized aza-PLADIPYs were tested at 25, 50, 100, and 250 μM. Cell viability was measured after 24 hours (FIG. 5). Cisplatin caused significant cell death, with viability dropping to 59, 56, 44, and 24%, at the respective concentrations. In contrast, all the tested aza-PLADIPYs displayed statistically, significantly less cytotoxicity than cisplatin. Compound 4, our aza-PLADIPY with the highest cytotoxicity in cancer cells, maintained cell viability of 100, 95, 90, and 71%, at the four respective concentrations. Compounds 1 and 2 also gave very high cell viability of ~100% at 100 μM and 88% at 250 M. These results suggest that the synthesized aza-PLADIPYs caused minimal damage to kidney cells up to a concentration of 100 μM. Even at 250 μM, their cytotoxicity was nearly a third of cisplatin.

In the effort to develop a dual acting anticancer agent, we synthesized five aza-PLADIPYs. These compounds displayed an unexpected Pt—C bond. All were tested for their cytotoxicity on OVCAR5 cells under both light and dark conditions. Unfortunately, these compounds did not show selectivity for light vs dark conditions, indicating an inability to serve as potential PDT agents. Although, no light selectivity was observed, they were able to produce cytotoxicity in OVCAR5 cells. Compounds 1 and 2 produced cell death equivalents to cisplatin, whereas Compound 4 surpassed it. These three compounds possess donating groups at the R position, suggesting that additional analogs with varying electronic properties at the para position should be synthesized and tested.

The synthesized aza-PLADIPYs were also tested on HK-2 kidney proximal tubule cells to assess nephrotoxicity. Impressively, these displayed a significant advantage in terms of reducing kidney toxicity. Overall, these novel aza-PLADIPYs show promise as anticancer agents, with efficacy comparable to cisplatin, while minimizing nephrotoxicity, a significant side effect of the DNA crosslinking agents. This makes these aza-PLADIPYs attractive as anticancer chemotherapy agents.

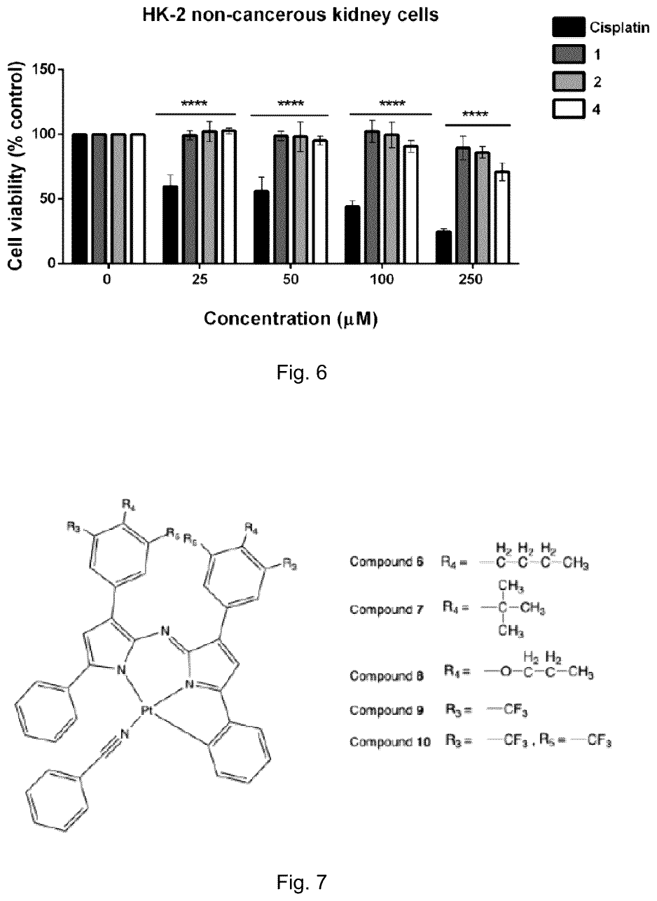

Synthesis of compounds 6-9 was carried out using the same method described in section 3.2. Initially, chalcones C6-C10 were synthesized by reacting substituted benzaldehyde with acetophenone. Next, these chalcones were reacted with nitromethane to yield the respective Michael adducts M6-M10. The Michael adducts were then treated with ammonium acetate to form the azadipyrromethanes A6-A10. Finally, the azadipyrromethanes were complexed with platinum using potassium tetrachloroplatinate (II) to achieve the desired aza-PLADIPYs (FIG. 6). The final purification of these compounds was a considerably challenging. Initially, purification using an 80 g silica gel RediSep gold column for a crude mass of approximately 120-200 mg was performed. This method utilized a gradient elution system with dichloromethane (DCM) and hexane, increasing from 10% to 100% DCM over an hour. However, this approach failed to yield sufficiently pure product. Subsequent efforts to improve purity involved modifying the gradient by extending the time frame and using various fixed ratios of hexane to DCM. Unfortunately, these adjustments did not significantly enhance the purity of the compounds.

As a result, the use of larger columns was explored. The most effective results were achieved with 240 g columns. The purification process was then conducted in stages, utilizing a series of gradient elutions, starting with 0-20% DCM for one column volume, followed by 20-40%, then 60-80%, and finally 80-100% DCM. This revised method produced substantially improved results in terms of compound purity with yields ranging from 10-30%.

Various modifications to the non-leaving groups of cisplatin were explored. Compounds 6-8 were synthesized with electron-donating groups attached to the aromatic ring. Furthermore, to evaluate the impact of electron-withdrawing groups, we synthesized compounds 9 and 10 (FIG. 7).

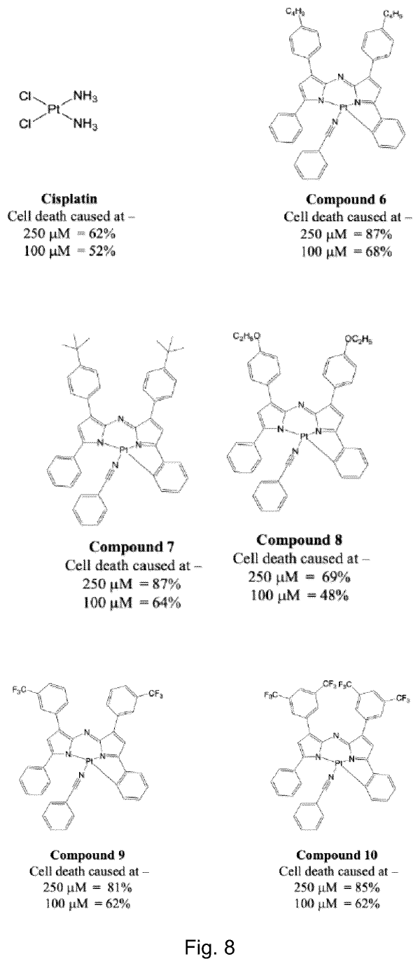

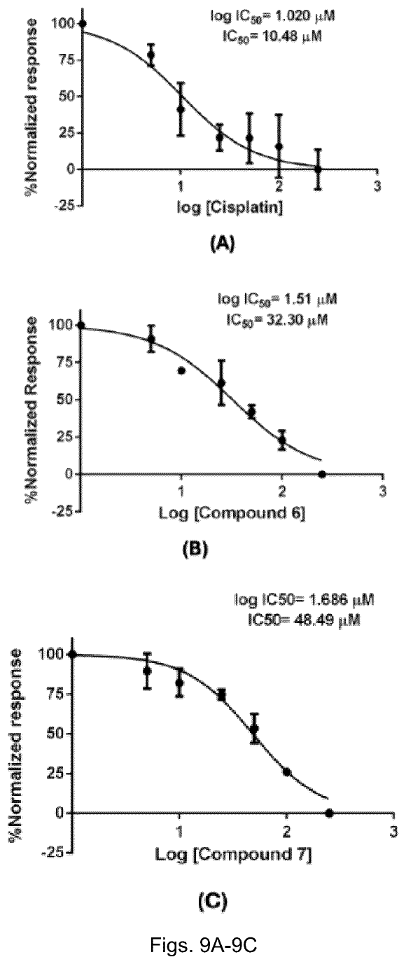

Initially, all the synthesized compounds were screened at two concentrations on the partially cisplatin-resistant OVCAR 5 cell line, using the same concentrations as in our previous work-100 μM and 250 μM.⁸⁰ Results are shown in FIG. 8. During this screening, cisplatin induced 62% cell death at 250 μM and 52% at 100 μM. Notably, all the newly synthesized aza-PLADIPY analogs exhibited greater cytotoxicity than cisplatin. Compounds 6 and 7, which contained electron-donating as well as lipophilic substituents, induced 87% cell death at 250 μM and at 100 μM a cell death of 68% and 64% respectively was observed. As a result of this, two compounds that displayed the greatest activity were selected to obtain a more detailed IC₅₀ value. Cell death data were obtained at 250, 100, 50, 10 and 5 μM from which an IC₅₀ value of 10.48 μM was obtained for cisplatin, 32.3 μM for compound 6 and 48.49 μM for compound 7 (FIGS. 9A-9C).

All newly synthesized compounds whether having electron-donating or withdrawing groups on the aromatic rings demonstrated superior cell death compared to cisplatin at concentrations of 100 μM and 2500 μM in the OVCAR5 cell line. Compounds 6 and 7, which induced the highest cell death, were selected for a detailed IC₅₀ analysis. Their calculated IC₅₀ values (32.3 M for Compound 6 and 48.49 μM for Compound 7) were higher than cisplatin's IC₅₀ of 10.48 μM. Despite this, the compounds may offer a critical renal safety advantage over cisplatin. Prior evaluations of the aza-PLADIPY scaffold indicate a significantly reduced risk of nephrotoxicity—a major dose-limiting side effect of cisplatin.⁸⁰ Earlier analogs preserved 70% viability in human HK-2 kidney cells even at 250 μM, starkly contrasting cisplatin's 24% viability at the same concentration. This improved safety profile could allow higher therapeutic doses without kidney damage risks. Notably, FDA-approved cisplatin analogs such as carboplatin were developed to reduce nephrotoxicity but require about 3 times higher doses than cisplatin.⁸¹ However, carboplatin still produces kidney damage and can induce mild forms of acute kidney injury.⁸² In comparison, while the two novel aza-PLADIPYs exhibit a slightly higher increase in IC₅₀ relative to cisplatin, prior work suggest that they will produce very minimal nephrotoxicity at those concentrations.⁸⁰

The present aza-PLADIPY compounds can be used to treat a wide variety of cancers. For example, they can be used to selectively kill cancer cells in a mammalian subject, such as a human or non-human mammalian subject, having a cancer selected from testicular cancer, ovarian cancer, bladder cancer, lung cancer, head and neck cancer, endometrial cancer, gastric cancer, esophageal cancer, breast cancer, cervical cancer, prostate cancer, neuroblastoma, multiple myeloma, melanoma, mesothelioma, leukemia, non-Hodgkin's lymphoma, and sarcomas. Preferred is treatment of cancers that have up to now been treated with cisplatin, such as advanced testicular cancer, ovarian cancer, bladder cancer, lung cancer (small cell and non-small cell) head and neck cancers, cervical cancer, breast cancer, gastric cancers, sarcomas, lymphomas, and malignant mesothelioma.

Specific aza-PLADIPY compounds can be tested for their efficacy against certain cancers using cell culture models of those cancers. For example, efficacy against testicular cancer can be tested using cell lines 833K, SuSa, GCT27, NTERA-2, and NCCIT. Efficacy against ovarian cancer can be tested using cell lines: A2780, A2780-CP20, IGROV1, OVCAR-3, and SKOV-3. Efficacy against breast cancer can be tested using cell lines MCF-7 (luminal), MDA-MB-231 (triple-negative), MDA-MB-468 (basal-like), and BT-549. Effectiveness against lung cancer can be tested against cell lines H23, H460, A549, and H1299. Head and neck or oral squamous cell carcinoma can be tested using cell lines YD-8, YD-9, YD-38, HN-3, and SCC-25. Bladder cancer can be represented by cell lines RT112, 5637, and T24. Other cancer models, including animal models, are described in the literature and can also be used with the present compounds.

Potency of the aza-PLADIPY compounds can be assessed by performing a dose-response experiment and determining the IC₅₀ for each compound. In general, the aza-PLADIPY compounds have an IC₅₀ for causing cell death of <300, <250, <200, <150, <100, <50, <40, <30, <20, <10, or <5, or <1 μM.

Possible side effects from the use of aza-PLADIPY compounds, such as unwanted toxicity to normal cells, tissues, and organs, also can be assessed using cultured cell lines. For example, nephrotoxicity can be assessed using HK-2 kidney proximal tubule cells, or any of a variety of animal models of kidney function. As an example, aza-PLADIPY compounds may exhibit less than 50%, less than 40%, less than 30%, less than 20%, less than 10%, less than 5%, less than 2%, or less than 1% cell death using a kidney cell line such as HK-2, and using a physiologically relevant concentration of the aza-PLADIPY compound, such as about 250, 200, 150, 100, 50, 20, 10, 5, 2, or 1 μM.

### EXAMPLES

### Example 1. Synthesis of Chalcone C6

Acetophenone (1 eq) was dissolved in absolute ethanol (10 ml). 30 ml of aqueous solution of 10% sodium hydroxide was added to it and stirred for 15 minutes after which a solution of 4-butylbenzaldehyde (1 eq) was added to it. The reaction was stirred for 24 hours during which the product precipitated forming an oily residue which was extracted using ethyl acetate to yield the desired chalcone C6. The product was used without purification for the next step. (5.05 g, 77% yield). ¹H NMR (400 MHz, CDCl₃) δ 8.04-7.97 (m, 1H), 7.79 (d, J=15.7 Hz, 1H), 7.58-7.43 (m, 4H), 7.24-7.16 (m, 1H), 2.61 (dt, J=4.2, 2.1 Hz, 1H), 1.59 (dddd, J=10.6, 7.9, 5.3, 2.6 Hz, 1H), 1.35 (dd, J=7.5, 2.4 Hz, 1H), 0.92 (td, J=7.4, 1.3 Hz, 2H). ¹³C NMR (101 MHz, CDCl₃) δ 190.69, 190.65, 146.15, 145.05, 138.38, 132.71, 132.38, 129.10, 128.62, 128.57, 128.51, 121.12, 121.10, 77.40, 77.35, 77.20, 77.14, 76.86, 76.82, 35.65, 33.39, 22.38, 13.98.

### Example 2. Synthesis of the Micheals Adduct M6

The synthesized chalcone C6 (1 eq) was mixed together with nitromethane (20 eq) and NaOH (0.2 eq) in 1 mL of absolute ethanol. The reaction mixture was heated at 60 °C for 12 hours after which it was cooled and concentrated to obtain an oily residue. This residue was then dissolved in ethyl acetate and washed with 50 mL water three times. After which, the organic layer was washed with brine, dried over magnesium sulfate, filtered and evaporated to yield the desired oily residue M6. The product was used without purification for the next step. (3.87 g, 32% yield). ¹H NMR (400 MHz, CDCl₃) δ 8.00-7.88 (m, 2H), 7.61-7.50 (m, 1H), 7.50-7.39 (m, 2H), 7.21-7.04 (m, 4H), 4.81 (dd, J=12.4, 6.7 Hz, 1H), 4.67 (dd, J=12.4, 7.9 Hz, 1H), 4.25-4.13 (m, 1H), 3.52-3.34 (m, 2H), 2.65-2.52 (m, 2H), 1.62-1.50 (m, 2H), 1.33 (h, J=7.3 Hz, 2H), 0.91 (t, J=7.3 Hz, 3H). ¹³C NMR (101 MHz, CDCl₃) δ 197.08, 170.95, 142.26, 136.67, 136.48, 133.40, 128.92, 128.65, 128.56, 127.99, 127.42, 79.66, 62.29, 60.26, 41.57, 38.98, 35.15, 33.44, 22.35, 20.76, 14.10, 13.87.

### Example 3. Synthesis of Azadipyrromethane A6

The Michael adduct M6 (1 eq) and ammonium acetate (35 eq) was added in 20 ml of butanol and heated to 110 °C for 24 hours. After 24 hours, the butanol was evaporated to approximately a quarter of its original volume, filtered and washed with absolute ethanol to yield the desired azapyrromethane solid A6. The product was used without purification for the next step. (336 mg, 6% yield). ¹H NMR (400 MHz, CDCl₃) δ 8.02-7.91 (m, 6H), 7.57-7.45 (m, 6H), 7.51-7.39 (m, 4H), 7.42-7.18 (m, 9H), 7.16 (s, 3H), 2.69 (t, J=7.7 Hz, 5H), 1.98 (s, 1H), 1.73-1.59 (m, 5H), 1.49-1.32 (m, 6H), 0.97 (td, J=7.3, 3.9 Hz, 9H). ¹³C NMR (101 MHz, CDCl₃) δ 155.01, 149.62, 142.98, 142.76, 132.31, 131.20, 129.95, 129.12, 129.02, 128.70, 128.39, 127.14, 127.03, 126.53, 114.31, 77.23, 35.55, 33.63, 22.38, 14.02, 13.97. mp: 208.6-209.9 °C.

### Example 4. Synthesis of Cyclometalated Platinum Azadipyrromethene 6

The azadipyrromethene ligand A6 (1 eq) was stirred with K₂PtCl₄ (1.2 eq), in PhCN (10 mL), at 140 °C for 48 hrs. The PhCN was then mostly removed under vacuum, and the dark purple residue was rinsed with hexane to remove residual PhCN. The crude product was purified by silica gel column chromatography to afford dark purple solid, 6. (45.6 mg, 17% yield). ¹H NMR (400 MHz, CDCl₃) δ 7.95-7.84 (m, 3H), 7.81 (d, J=8.1 Hz, 1H), 7.41 (t, J=7.9 Hz, 1H), 7.35-7.25 (m, 5H), 7.19 (dd, J=14.9, 8.0 Hz, 3H), 7.05 (dd, J=7.4, 1.3 Hz, 1H), 2.68 (q, J=7.7 Hz, 3H), 1.73-1.59 (m, 3H), 1.41 (hd, J=7.3, 4.7 Hz, 3H), 0.96 (td, J=7.3, 2.6 Hz, 4H). ¹³C NMR (101 MHz, CDCl₃) δ 143.13, 142.87, 133.82, 132.57, 131.84, 131.44, 130.65, 129.80, 129.13, 128.97, 128.94, 128.80, 128.15, 128.04, 127.86, 124.21, 118.24, 109.90, 77.23, 35.52, 33.70, 33.65, 22.36, 14.01. mp: 211.08 °C.

### Example 5. Synthesis of Chalcone C7

Acetophenone (1 eq) was dissolved in absolute ethanol (10 ml). 30 ml of aqueous solution of 10% sodium hydroxide was added to it and stirred for 15 minutes after which a solution of 4-tert-butyl benzaldehyde (1 eq) was added to it. The reaction was stirred for 24 hours during which the product precipitated from the solution and was collected using vacuum filtration. The solid was then washed with a cold, 50:50 mixture of ethanol and water to yield the desired chalcone C7. The product was used without purification for the next step. (4.8 g, 73% yield). ¹H NMR (400 MHz, CDCl₃) δ 8.10-8.03 (m, 2H), 7.86 (d, J=15.7 Hz, 1H), 7.64-7.38 (m, 8H), 1.38-1.26 (m, 9H). ¹³C NMR (101 MHz, CDCl₃) δ 190.52, 190.48, 154.20, 154.18, 144.85, 144.83, 138.40, 132.74, 132.19, 128.66, 128.60, 128.56, 128.46, 128.35, 125.99, 121.29, 77.55, 77.02, 34.95, 31.33, 31.21, 31.10. mp: 101-103.2 °C.

### Example 6. Synthesis of the Micheals Adduct M7

The synthesized chalcone C7 (1 eq) was mixed together with nitromethane (20 eq) and NaOH (0.2 eq) in 1 mL of absolute ethanol. The reaction mixture was heated at 60 °C for 12 hours after which it was cooled and concentrated to obtain an oily residue. This residue was then dissolved in ethyl acetate and washed with 50 mL water three times. After which, the organic layer was washed with brine, dried over magnesium sulfate, filtered and evaporated to yield the desired oily residue M7. The product was used without purification for the next step. (4.2 g, 37% yield). ¹H NMR (400 MHz, CDCl₃) δ 8.00-7.91 (m, 2H), 7.53-7.42 (m, 2H), 7.40-7.32 (m, 2H), 7.26-7.19 (m, 2H), 4.84 (dd, J=12.4, 6.7 Hz, 3H), 4.74 (d, J=4.0 Hz, 43H), 3.55-3.37 (m, 2H), 1.31 (s, 9H). ¹³C NMR (101 MHz, CDCl₃) δ 196.99, 171.06, 150.58, 136.43, 136.12, 133.44, 128.66, 127.99, 127.08, 125.88, 79.52, 62.43, 60.32, 41.62, 38.74, 34.42, 31.20, 29.66, 20.94, 14.13.

### Example 7. Synthesis of Azadipyrromethane A7

The Michael adduct M7 (1 eq) and ammonium acetate (35 eq) was added in 20 ml of butanol and heated to 110 °C for 24 hours. After 24 hours, the butanol was evaporated to approximately a quarter of its original volume, filtered and washed with absolute ethanol to yield the desired azapyrromethane solid A7. The product was used without purification for the next step. (560 mg, 9% yield). ¹H NMR (400 MHz, CDCl₃) δ 8.27-8.20 (m, 1H), 8.07-7.95 (m, 7H), 7.91 (d, J=10.2 Hz, 1H), 7.73 (dd, J=8.5, 3.0 Hz, 1H), 7.63-7.46 (m, 13H), 7.46-7.36 (m, 2H), 7.20 (s, 2H), 2.01 (d, J=2.2 Hz, 1H), 1.42 (d, J=1.5 Hz, 18H), 1.40-1.23 (m, 11H), 1.03-0.92 (m, 1H). ¹³C NMR (101 MHz, CDCl₃) δ 155.10, 151.07, 149.58, 142.72, 132.33, 130.99, 129.97, 129.12, 128.98, 128.71, 127.14, 126.88, 126.54, 125.15, 114.30, 77.23, 34.68, 31.33. mp: 248.1-249.2 °C.

### Example 8. Synthesis of Cyclometalated Platinum Azadipyrromethene 7

The azadipyrromethene ligand A7 (1 eq) was stirred with K₂PtCl₄ (1.2 eq), in PhCN (10 mL), at 140 °C for 48 hrs. The PhCN was then mostly removed under vacuum, and the dark purple residue was rinsed with hexane to remove residual PhCN. The crude product was purified by silica gel column chromatography to afford dark purple solid, 7. (34 mg, 10% yield). ¹H NMR ¹H NMR (400 MHz, CDCl₃) δ 8.02-7.94 (m, 1H), 7.94-7.82 (m, 2H), 7.77-7.63 (m, 1H), 7.52-7.38 (m, 3H), 7.42-7.28 (m, 3H), 7.11-7.03 (m, 1H), 7.07-6.85 (m, 1H), 6.80 (s, OH), 1.40 (d, J=8.6 Hz, 10H), 1.40-1.20 (m, 1H), 1.29 (s, 1H). ¹³C NMR (101 MHz, CDCl₃) δ 22.36, 33.65, 33.70, 35.52 77.23, 109.90 118.28, 124.21, 127.86, 128.04, 128.15 128.80, 128.94, 128.97, 129.13, 129.80, 130.65, 131.44, 131.84, 132.57, 133.82, 142.87, 143.13. mp: 276.56 °C.

### Example 9. Synthesis of Chalcone C8

Acetophenone (1 eq) was dissolved in absolute ethanol (10 ml). 30 ml of aqueous solution of 10% sodium hydroxide was added to it and stirred for 15 minutes after which a solution of 4-ethoxybenzaldehyde (1 eq) was added to it. The reaction was stirred for 24 hours during which the oily product precipitated forming the solution which was extracted using ethyl acetate to yield the desired oily chalcone C8. The product was used without purification for the next step. (4.8 g, 88% yield). ¹H NMR (400 MHz, CDCl₃) δ 8.07-8.00 (m, 2H), 7.81 (d, J=15.6 Hz, 1H), 7.65-7.48 (m, 5H), 7.44 (d, J=15.6 Hz, 1H), 6.98-6.92 (m, 2H), 4.11 (q, J=7.0 Hz, 2H), 1.47 (t, J=7.0 Hz, 3H).¹³C (101 MHz, CDCl₃) δ 190.63, 161.13, 144.81, 138.57, 133.12, 132.54, 130.26, 128.58, 128.43, 128.33, 128.17, 127.46, 119.69, 114.91, 77.23, 63.68, 26.63, 14.75.

### Example 10. Synthesis of the Micheals Adduct M8

The synthesized chalcone C8 (1 eq) was mixed together with nitromethane (20 eq) and NaOH (0.2 eq) in 1 mL of absolute ethanol. The reaction mixture was heated at 60 °C for 12 hours after which it was cooled and concentrated to obtain an oily residue. This residue was then dissolved in ethyl acetate and washed with 50 mL water three times. After which, the organic layer was washed with brine, dried over magnesium sulfate, filtered and evaporated to yield the desired oily residue M8. The product was used without purification for the next step. (4.5 g, 38% yield). ¹H NMR (400 MHz, CDCl₃) δ 8.02-7.90 (m, 5H), 7.64-7.52 (m, 3H), 7.48 (ddt, J=8.1, 6.5, 1.3 Hz, 5H), 6.82 (dd, J=13.3, 1.2 Hz, 7H), 4.83 (dd, J=12.4, 6.7 Hz, 2H), 4.69 (dd, J=12.3, 8.0 Hz, 2H), 4.25-4.09 (m, 4H), 3.87 (d, J=9.1 Hz, 16H), 3.48 (dd, J=17.5, 6.5 Hz, 2H), 3.45-3.29 (m, 2H), 2.06 (s, 3H), 1.28 (t, J=7.1 Hz, 3H). ¹³C (101 MHz, CDCl₃) δ 197.03, 158.49, 136.46, 133.54, 133.13, 133.06, 130.84, 128.75, 128.60, 128.50, 128.40, 128.33, 128.17, 128.04, 114.97, 114.56, 79.87, 77.36, 77.05, 76.73, 63.45, 45.19, 41.69, 38.66, 26.64, 14.82.

### Example 11. Synthesis of Azadipyrromethane A8

The Michael adduct M8 (1 eq) and ammonium acetate (35 eq) was added in 20 ml of butanol and heated to 110 °C for 24 hours. After 24 hours, the butanol was evaporated to approximately a quarter of its original volume, filtered and washed with absolute ethanol to yield the desired azapyrromethane solid A8. The product was used without purification for the next step. (450 mg, 5% yield). ¹H NMR (400 MHz, CDCl₃ (400 MHz, CDCl₃) δ 8.10-8.02 (m, 2H), 7.97 (dd, J=7.2, 1.8 Hz, 2H), 7.55 (dd, J=8.3, 6.7 Hz, 2H), 7.52-7.44 (m, 1H), 7.14 (s, 1H), 7.03-6.95 (m, 2H), 4.15 (q, J=7.0 Hz, 2H), 1.50 (t, J=7.0 Hz, 3H). ¹³C NMR (101 MHz, CDCl₃) δ 130.37, 129.10, 126.51, 114.34, 113.48, 77.22, 63.54, 14.89. mp: 255.9-256.8 °C.

### Example 12. Synthesis of Cyclometalated Platinum Azadipyrromethene 8

The azadipyrromethene ligand A8 (1 eq) was stirred with K₂PtCl₄ (1.2 eq), in PhCN (10 mL), at 140 °C for 48 hrs. The PhCN was then mostly removed under vacuum, and the dark purple residue was rinsed with hexane to remove residual PhCN. The crude product was purified by silica gel column chromatography to afford dark purple solid, 8. (40 mg, 11% yield). ¹H NMR (400 MHz, CDCl₃) δ 7.95-7.84 (m, 3H), 7.81 (d, J=8.1 Hz, 1H), 7.41 (t, J=7.9 Hz, 1H), 7.35-7.25 (m, 5H), 7.19 (dd, J=14.9, 8.0 Hz, 3H), 7.05 (dd, J=7.4, 1.3 Hz, 1H), 2.68 (q, J=7.7 Hz, 3H), 1.73-1.59 (m, 3H), 1.41 (hd, J=7.3, 4.7 Hz, 3H), 0.96 (td, J=7.3, 2.6 Hz, 4H). ¹³C NMR (101 MHz, CDCl₃) δ 166.12, 159.31, 159.15, 147.78, 145.05, 143.56, 141.78, 136.11, 133.77, 132.60, 132.56, 132.02, 131.82, 131.73, 131.14, 130.99, 130.75, 130.56, 130.38, 129.07, 128.92, 128.75, 127.86, 127.57, 126.84, 126.61, 125.01, 124.14, 117.49, 114.13, 114.02, 113.78, 113.61, 109.10, 77.24, 63.50, 14.91. mp: 238.86°.

### Example 13. Synthesis of Chalcone C9

Acetophenone (1 eq) was dissolved in absolute ethanol (10 ml). 30 ml of aqueous solution of 10% sodium hydroxide was added to it and stirred for 15 minutes after which a solution of 3-(trifluoromethyl)benzaldehyde (1 eq) was added to it. The reaction was stirred for 24 hours during which the product precipitated from the solution and was collected using vacuum filtration. The solid was then washed with a cold, 50:50 mixture of ethanol and water to yield the desired chalcone C9. The product was used without purification for the next step. (3.8 g, 92% yield). ¹H NMR (400 MHz, CDCl₃) δ 8.04-7.97 (m, 1H), 7.79 (d, J=15.7 Hz, 1H), 7.58-7.43 (m, 4H), 7.24-7.16 (m, 1H), 2.61 (dt, J=4.2, 2.1 Hz, 1H), 1.59 (dddd, J=10.6, 7.9, 5.3, 2.6 Hz, 1H), 1.35 (dd, J=7.5, 2.4 Hz, 1H), 0.92 (td, J=7.4, 1.3 Hz, 2H). ¹³C NMR (101 MHz, CDCl₃) δ 190.69, 190.65, 146.15, 145.05, 138.38, 132.71, 132.38, 129.10, 128.62, 128.57, 128.51, 121.12, 121.10, 77.40, 77.35, 77.20, 77.14, 76.86, 76.82, 35.65, 33.39, 22.38, 13.98. mp: 104.2-105.9 °C.

### Example 14. Synthesis of the Micheals Adduct M9

The synthesized chalcone C9 (1 eq) was mixed together with nitromethane (20 eq) and NaOH (0.2 eq) in 1 mL of absolute ethanol. The reaction mixture was heated at 60 °C for 12 hours after which it was cooled and concentrated to obtain an oily residue. This residue was then dissolved in ethyl acetate and washed with 50 mL water three times. After which, the organic layer was washed with brine, dried over magnesium sulfate, filtered and evaporated to yield the desired oily residue M9. The product was used without purification for the next step. (2.99 g, 34% yield). ¹H NMR (400 MHz, CDCl₃) δ 8.00-7.88 (m, 2H), 7.61-7.50 (m, 1H), 7.50-7.39 (m, 2H), 7.21-7.04 (m, 4H), 4.81 (dd, J=12.4, 6.7 Hz, 1H), 4.67 (dd, J=12.4, 7.9 Hz, 1H), 4.25-4.13 (m, 1H), 3.52-3.34 (m, 2H), 2.65-2.52 (m, 2H), 1.62-1.50 (m, 2H), 1.33 (h, J=7.3 Hz, 2H), 0.91 (t, J=7.3 Hz, 3H). ¹³C NMR (101 MHz, CDCl₃) δ 195.74, 140.03, 139.02, 134.72, 129.48, 129.13, 129.08, 127.97, 127.48, 79.54, 41.51, 39.28.

### Example 15. Synthesis of Azadipyrromethane A9

The Michael adduct M9 (1 eq) and ammonium acetate (35 eq) was added in 20 ml of butanol and heated to 110 °C for 24 hours. After 24 hours, the butanol was evaporated to approximately a quarter of its original volume, filtered and washed with absolute ethanol to yield the desired azapyrromethane solid A9. The product was used without purification for the next step. (280 mg, 5% yield). ¹H NMR (400 MHz, CDCl₃) δ 8.02-7.91 (m, 6H), 7.57-7.45 (m, 6H), 7.51-7.39 (m, 4H), 7.42-7.18 (m, 9H), 7.16 (s, 3H), 2.69 (t, J=7.7 Hz, 5H), 1.98 (s, 1H), 1.73-1.59 (m, 5H), 1.49-1.32 (m, 6H), 0.97 (td, J=7.3, 3.9 Hz, 9H). ¹³C NMR (101 MHz, CDCl₃) δ 155.01, 149.62, 142.98, 142.76, 132.31, 131.20, 129.95, 129.12, 129.02, 128.70, 128.39, 127.14, 127.03, 126.53, 114.31, 77.23, 35.55, 33.63, 22.38, 14.02, 13.97. mp: 192-194.2 °C.

### Example 16. Synthesis of Cyclometalated Platinum Azadipyrromethene 9

The azadipyrromethene ligand A9 (1 eq) was stirred with K₂PtCl₄ (1.2 eq), in PhCN (10 mL), at 140 °C for 48 hrs. The PhCN was then mostly removed under vacuum, and the dark purple residue was rinsed with hexane to remove residual PhCN. The crude product was purified by silica gel column chromatography to afford dark purple solid, 9. (60 mg, 33% yield). ¹H NMR (400 MHz, CDCl₃) δ 7.95-7.84 (m, 3H), 7.81 (d, J=8.1 Hz, 1H), 7.41 (t, J=7.9 Hz, 1H), 7.35-7.25 (m, 5H), 7.19 (dd, J=14.9, 8.0 Hz, 3H), 7.05 (dd, J=7.4, 1.3 Hz, 1H), 2.68 (q, J=7.7 Hz, 3H), 1.73-1.59 (m, 3H), 1.41 (hd, J=7.3, 4.7 Hz, 3H), 0.96 (td, J=7.3, 2.6 Hz, 4H). ¹³C NMR (101 MHz, CDCl₃) δ 143.13, 142.87, 133.82, 132.57, 131.84, 131.44, 130.65, 129.80, 129.13, 128.97, 128.94, 128.80, 128.15, 128.04, 127.86, 124.21, 118.24, 109.90, 77.23, 35.52, 33.70, 33.65, 22.36, 14.01. mp: 270.72 °C.

### Example 17. Synthesis of Chalcone C10

Acetophenone (1 eq) was dissolved in absolute ethanol (10 ml). 30 ml of aqueous solution of 10% sodium hydroxide was added to it and stirred for 15 minutes after which a solution of 3,5-bis(trifluoromethyl)benzaldehyde (1 eq) was added to it. The reaction was stirred for 24 hours during which the product precipitated from the solution and was collected using vacuum filtration. The solid was then washed with a cold, 50:50 mixture of ethanol and water to yield the desired chalcone C10. The product was used without purification for the next step. (4.14 g, 72% yield). ¹H NMR (400 MHz, CDCl₃) δ 8.03-7.94 (m, 6H), 7.78 (ddd, J=7.8, 5.7, 2.3 Hz, 1H), 7.66-7.57 (m, 1H), 7.58 (s, 1H), 7.59-7.54 (m, 1H), 7.57-7.48 (m, 1H), 7.52-7.43 (m, 5H), 7.47-7.37 (m, 3H), 7.41-7.28 (m, 2H), 7.32-7.22 (m, 4H), 7.25-7.13 (m, 1H), 4.45 (p, J=7.0 Hz, 1H), 3.61-3.37 (m, 5H), 1.35-1.23 (m, 1H). ¹³C NMR (101 MHz, CDCl₃) δ 198.21, 147.94, 147.31, 136.70, 136.41, 135.49, 133.20, 131.54, 129.19, 128.77, 128.63, 128.47, 128.27, 128.23, 128.21, 128.13, 128.08, 127.94, 127.86, 127.82, 127.72, 127.21, 127.16, 126.76, 124.38, 121.92, 121.41, 120.02, 119.80, 119.26, 77.25, 45.86, 43.34, 31.77, 31.48. mp: 67.2-68.3 °C.

### Example 18. Synthesis of the Micheals Adduct M10

The synthesized chalcone C10 (1 eq) was mixed together with nitromethane (20 eq) and NaOH (0.2 eq) in 1 mL of absolute ethanol. The reaction mixture was heated at 60 °C for 12 hours after which it was cooled and concentrated to obtain an oily residue. This residue was then dissolved in ethyl acetate and washed with 50 mL water three times. After which, the organic layer was washed with brine, dried over magnesium sulfate, filtered and evaporated to yield the desired oily residue M10. The product was used without purification for the next step. (4.08 g, 54% yield). ¹H NMR (400 MHz, CDCl₃) δ 8.03-7.92 (m, 2H), 7.69-7.54 (m, 1H), 7.54-7.44 (m, 2H), 7.41-7.30 (m, 3H), 7.33-7.21 (m, 1H), 4.92-4.76 (m, 2H), 4.57 (ddd, J=13.8, 7.5, 6.3 Hz, 1H), 3.62-3.37 (m, 2H). ¹³C NMR (101 MHz, CDCl₃) δ 196.57, 147.43, 136.20, 133.70, 130.83, 129.27, 128.80, 128.55, 128.26, 128.03, 127.05, 121.85, 120.16, 120.14, 120.12, 119.28, 77.90, 40.25, 33.86.

### Example 19. Synthesis of Azadipyrromethane A10

The Michael adduct M10 (1 eq) and ammonium acetate (35 eq) was added in 20 ml of butanol and heated to 110 °C for 24 hours. After 24 hours, the butanol was evaporated to approximately a quarter of its original volume, filtered and washed with absolute ethanol to yield the desired azapyrromethane solid A10. The product was used without purification for the next step. (1.3 g, 14% yield). ¹H NMR (400 MHz, CDCl₃) δ 8.11 (dt, J=7.8, 0.9 Hz, 1H), 8.01-7.94 (m, 2H), 7.62-7.54 (m, 2H), 7.58-7.47 (m, 2H), 7.40-7.34 (m, 2H), 7.37 (s, 2H), 7.38-7.24 (m, 1H), 1.28 (s, 7H), 0.94-0.83 (m, 2H), 0.88 (s, 2H). ¹³C NMR (101 MHz, CDCl₃) δ 155.24, 149.97, 147.01, 136.74, 133.71, 132.06, 130.28, 129.24, 128.77, 126.81, 126.65, 126.12, 120.64, 118.68, 77.22, 29.72. mp: 199.3-202 °C.

### Example 20. Synthesis of Cyclometalated Platinum Azadipyrromethene 10

The azadipyrromethene ligand A10 (1 eq) was stirred with K₂PtCl₄ (1.2 eq), in PhCN (10 mL), at 140 °C for 48 hrs. The PhCN was then mostly removed under vacuum, and the dark purple residue was rinsed with hexane to remove residual PhCN. The crude product was purified by silica gel column chromatography to afford dark purple solid, 10. (88.6 mg, 18% yield). ¹H NMR (400 MHz, CDCl₃) δ 8.08 (dd, J=7.9, 1.7 Hz, 1H), 7.89 (dt, J=8.6, 1.6 Hz, 3H), 7.73-7.64 (m, 1H), 7.54-7.42 (m, 2H), 7.40-7.32 (m, 5H), 7.32 (dt, J=4.7, 1.6 Hz, 3H), 7.32-7.20 (m, 2H), 7.20-6.88 (m, 5H). ¹³C NMR (101 MHz, CDCl₃) δ 147.18, 135.58, 134.60, 133.96, 133.63, 132.61, 131.87, 130.86, 129.48, 129.06, 128.97, 128.60, 128.48, 127.91, 125.95, 125.75, 125.52, 124.38, 122.31, 120.43, 77.23, 29.73. mp: 203.31 °C.

### Example 21. Cell Viability Assay

Human NIH: OVCAR5 cell line was purchased from Fox Chase Cancer Center (Philadelphia, PA). This cell line was maintained in RPMI 1640 Medium (Gibco™, 61-870-127) supplemented with 10% premium grade FBS (R&D Systems, S11150), and 1% penicillin/streptomycin (Gibco™, 15-140-122). The CHO-K1 cell line was maintained in F-12 K Medium (ATCC, 30-2004) supplemented with 10% premium grade FBS (R&D Systems, S11150), and 1% penicillin/streptomycin (Gibco™, 15-140-122). All immortalized cell lines were cultured in a humidified incubator at 5% CO₂ and 37 °C.⁷¹

Cells were plated in lack-walled optically clear bottom 96-well plates (PerkinElmer, 6055300; 4000 cells per well depending on the cell type) and incubated for 24 hours. After incubation, the media was aspirated and the cells were exposed to 100 and 250 μM of cisplatin, and compounds 6-10 for 24 hours for the initial screening. Whereas, to obtain a detailed IC₅₀, cells were exposed to cisplatin, compounds 6 and 7 at concentrations of 5, 10, 25, 50, 100, 250 μM for 24 hours. After incubation for 24 hours, cell viability was measured using the CellTiter-Glo® Luminescent Cell Viability Assay. The unfiltered luminescence was recorded by using a plate reader (BioTek, Synergy LX Multi-Mode Reader) at 1 sec integration time.

As used herein, “consisting essentially of” allows the inclusion of materials or steps that do not materially affect the basic and novel characteristics of the claim. Any recitation herein of the term “comprising”, particularly in a listing of components of a composition or elements of a device, constitutes inclusion of alternative embodiments in which “comprising” is replaced with “consisting essentially of” or “consisting of”.

While the present invention has been described in conjunction with certain preferred embodiments, one of ordinary skill, after reading the foregoing specification, will be able to effect various changes, substitutions of equivalents, and other alterations to the compositions and methods set forth herein.

### REFERENCES

1. Ostios-Garcia L, Pérez D M, Castelo B, et al. Classification of Anticancer Drugs: An update with FDA- and EMA-approved drugs. Cancer Metastasis Rev. 2024; 43(4):1561-1571. doi:10.1007/s10555-024-10188-5
2. Emadi A, Karp J E. Cancer Pharmacology: An Illustrated Manual of Anticancer Drugs. Demos Medical, an imprint of Springer Publishing; 2024.
3. Gunaydin G, Gedik M E, Ayan S. Photodynamic therapy-current limitations and novel approaches. Front Chem. 2021; 9:6917697. doi:10.3389/fchem.2021.691697
4. Gunaydin G, Gedik M E, Ayan S. Photodynamic therapy for the treatment and diagnosis of cancer—a review of the current clinical status. Front Chem. 2021; 9:686303. doi:10.3389/fchem.2021.686303
5. Broadwater D, Medeiros H C D, Lunt R R, Lunt S Y. Current advances in photoactive agents for cancer imaging and therapy. Annu Rev Biomed Eng. 2021; 23(1):29-60. doi:10.1146/annurev-bioeng-122019-115833
6. Kessel D. Photodynamic therapy: A brief history. J. Clin. Med. 2019; 8(10):1581. doi:10.3390/jcm8101581
7. Yang W, Sourvanos D, Lu B, et al. A comparison of multichannel singlet oxygen luminescence dosimetry (MSOLD) and singlet oxygen explicit dosimetry (SOED). Cancers. 2016; 8(12):109. doi:10.1117/12.3003421
8. Priefer R, R. Griffiths J, N. Ludwig J, Skelhorne-Gross G, S. Greene R. Synthesis of aza-bodipy boron difluoride PDT agents to promote apoptosis in Hela cells. Lett Org Chem. 2011; 8(6):368-373. doi:10.2174/157017811796064377
9. Makovec T. Cisplatin and beyond: Molecular mechanisms of action and drug resistance development in cancer chemotherapy. Radiol Oncol. 2019; 53(2):148-158. doi:10.2478/raon-2019-0018
10. Kishimoto T, Yoshikawa Y, Yoshikawa K, Komeda S. Different effects of Cisplatin and Transplatin on the higher-order structure of DNA and gene expression. Int J Mol Sci. 2019; 21(1):34. doi:10.3390/ijms21010034
11. Ravera M, Gabano E, Sardi M, et al. Synthesis, characterization, structure, molecular modeling studies and biological activity of sterically crowded pt(ii) complexes containing bis(imidazole) ligands. J Inorg Biochem. 2011; 105(3):400-409. doi:10.1016/j.jinorgbio.2010.12.002
12. Casas J S, Castiñeiras A, Parajó Y, et al. Pd(II) and Pt(II) complexes of 2,2′-biimidazole and its N,N′-dimethyl derivative. The crystal structure of [{PtBr(DMSO)}2(Me2bim)] (Me2bim=N,N′-dimethyl-2,2′-biimidazole). Polyhedron. 2003; 22(8):1113-1121. doi:10.1016/s0277-5387(03)00097-4
13. Crespo M. Diarylplatinum(ii) compounds as versatile metallating agents in the synthesis of cyclometallated platinum compounds with N-donor ligands. Inorganics. 2014; 2(1):115-131. doi:10.3390/inorganics2010115
14. Brooks J, Babayan Y, Lamansky S, et al. Synthesis and characterization of phosphorescent cyclometalated platinum complexes. Inorg Chem. 2002; 41(12):3055-3066. doi:10.1021/ic0255508
15. Kwong R C, Sibley S, Dubovoy T, Baldo M, Forrest S R, Thompson M E. Efficient, saturated red organic light emitting devices based on phosphorescent platinum(ii) porphyrins. Chem. Mater. 1999; 11(12):3709-3713. doi:10.1021/cm9906248
16. Manathanath M, Xie M, Arunkumar C, Wang Z, Zhao J, Sujatha S. Synthesis, photophysical, electrochemical and photoluminescent oxygen sensing studies of Trans-pt(ii)-porphyrins. Dyes Pigm. 2019; 165:117-127. doi:10.1016/j.dyepig.2019.02.016
17. Mason R, Robertson G B. Crystal structure of a diolefin complex of platinum(II). J. Chem. Soc. A. 1969:492-497. doi:10.1039/j19690000492
18. van Zyl B, Tang D, Bowden N A. Biomarkers of platinum resistance in ovarian cancer: What can we use to improve treatment. Endocr Relat Cancer. 2018; 25(5):303-318. doi:10.1530/erc-17-0336
19. Kleih M, Bopple K, Dong M, et al. Direct impact of cisplatin on mitochondria induces ROS production that dictates cell fate of ovarian cancer cells. Cell Death Dis. 2019; 10(11):851. doi:10.1038/s41419-019-2081-4
20. McSweeney K R, Gadanec L K, Qaradakhi T, Ali B A, Zulli A, Apostolopoulos V. Mechanisms of cisplatin-induced acute kidney injury: Pathological mechanisms, pharmacological interventions, and genetic mitigations. Cancers. 2021; 13(7):1572. doi:10.3390/cancers13071572
21. Oh G-S, Kim H-J, Shen A, et al. Cisplatin-induced kidney dysfunction and perspectives on improving treatment strategies. Electrolyte Blood Press. 2014; 12(2):55-56. doi:10.5049/ebp.2014.12.2.55
22. Krause L, Herbst-Irmer R, Sheldrick G M, Stalke D. Comparison of silver and molybdenum microfocus X-ray sources for single-crystal structure determination. J App/Crystallogr. 2015; 48(1):3-10. doi:10.1107/s1600576714022985

1. A compound of Formula I: 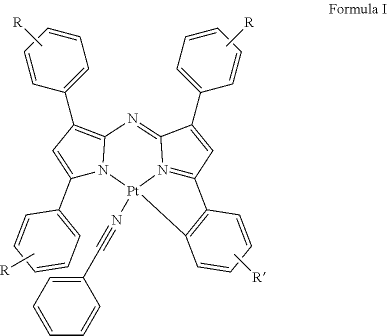
wherein R and R′ are independently selected from one or more substituents on a designated ring structure, the substituents selected from the group consisting of H, C1-C12 linear or branched alkyl, C1-C12 alkylamino, amino, hydroxy, C1-C12 hydroxyalkyl, C1-C12 thioalkyl, C1-C12 cycloalkyl, aryl, C1-C12 alkoxy, C1-C12 amide, halo, C1-C12 haloalkyl, CF₃, NO₂, cyano, C1-C12 carboxy, and C1-C12 sulfonate, wherein R and R′ are ortho, meta, or para.

2. The compound of claim 1, wherein R is methyl, methoxy, or chloro.

3. The compound of claim 1, wherein R and/or R′ is methoxy.

4. The compound of claim 1, wherein the compound is selected from the following compounds:

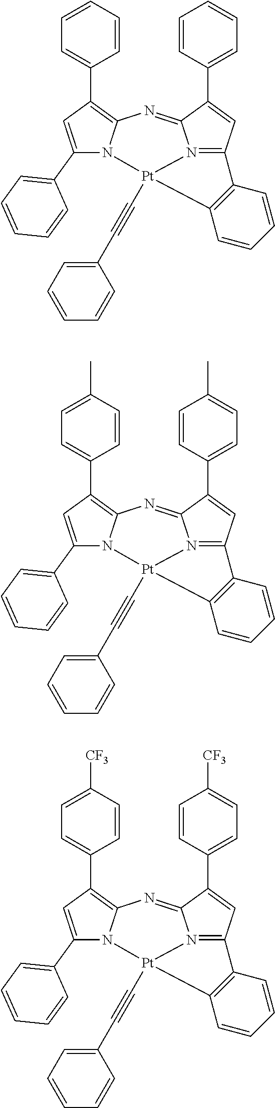

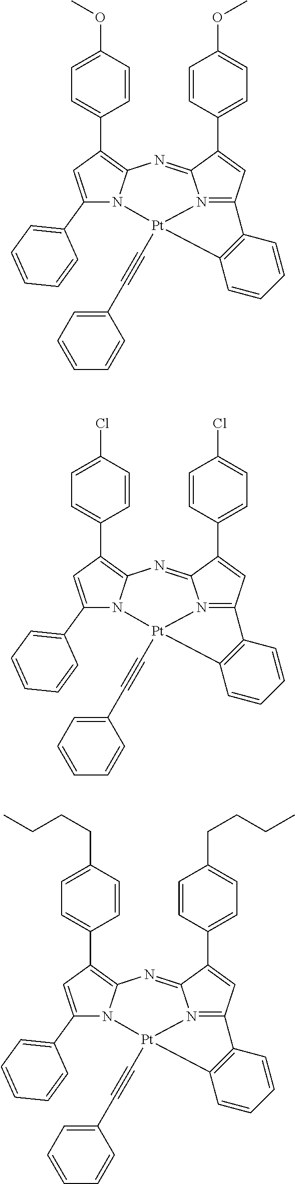

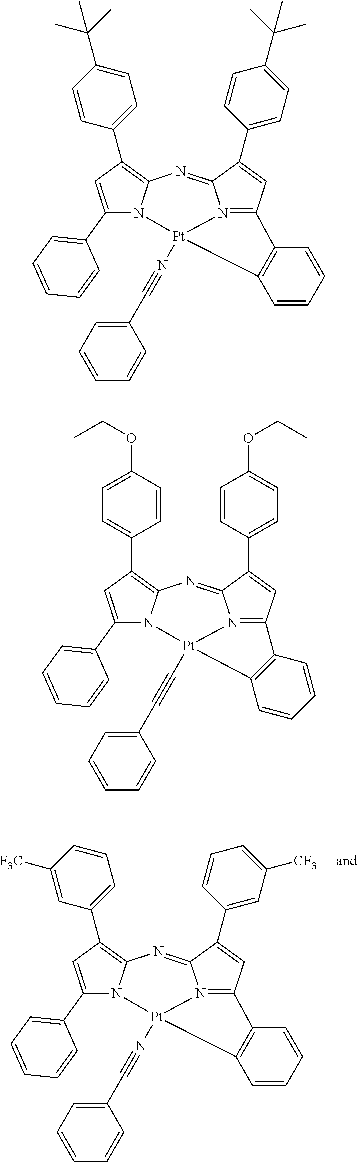

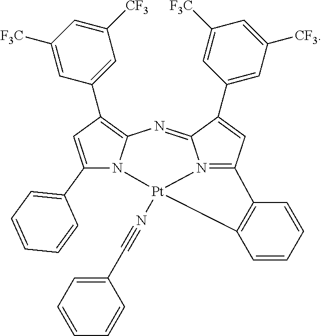

5. The compound of claim 1, wherein the compound is an anticancer agent.

6. The compound of claim 1, wherein the compound has a higher cytotoxicity toward cancer cells than cisplatin.

7. The compound of claim 1, wherein the compound kills at least 70% of cancer cells at a concentration of 250 μM.

8. The compound of claim 1, wherein the compound lacks nephrotoxicity at a concentration that produces at least 50% cell death of cancer cells.

9. A pharmaceutical composition comprising the compound of claim 1 and at least one excipient.

10. The pharmaceutical composition of claim 9, further comprising one or more additional antitumor agents.

11. The pharmaceutical composition of claim 10, wherein the one or more additional antitumor agents are selected from small molecule antitumor agents, aptamers and antibodies.

12. The pharmaceutical composition of claim 11, wherein the one or more additional antitumor agents are small molecules selected from the group consisting of alkylating agents, antimetabolites, antibiotics, taxanes, Vinca alkaloids, kinase inhibitors, and immune checkpoint inhibitors.

13. The pharmaceutical composition of claim 11, wherein the one or more additional antitumor agents are antibodies selected from the group consisting of rituximab, trastuzumab, bevacizumab, cetuximab.

14. The pharmaceutical composition of claim 11, wherein the one or more additional antitumor agents are antibodies selected from the group consisting of monoclonal antibodies, bispecific antibodies, and recombinant single chain antibodies.

15. A method of treating cancer in a mammalian subject, the method comprising administering to a mammalian subject in need thereof the compound of claim 1, whereby cancer cells in the subject are killed.

16. The method of claim 15, wherein said administering is not nephrotoxic.

17. The method of claim 15, wherein the cancer is selected from the group consisting of testicular cancer, ovarian cancer, bladder cancer, lung cancer, head and neck cancer, endometrial cancer, gastric cancer, esophageal cancer, breast cancer, cervical cancer, prostate cancer, neuroblastoma, multiple myeloma, melanoma, mesothelioma, leukemia, non-Hodgkin's lymphoma, and sarcomas.

18. The method of claim 15, wherein said compound or pharmaceutical composition is administered by intravenous administration.

19. The method of claim 15, further comprising administering radiation treatment to the mammalian subject.

20. A pharmaceutical kit or pack comprising the compound of claim 1 and another pharmaceutically active agent.
